(19) United States
(12) Patent Application Publication
(10) Pub. No.: US 2022/0265527 A1
(43) Pub. Date: Aug. 25, 2022

(54) MICROENCAPSULATED PROBIOTIC AND COMPOSITIONS CONTAINING THE SAME

(71) Applicant: GPCP IP Holdings LLC, Atlanta, GA (US)

(72) Inventors: Brian P. Argo, Greenville, WI (US); Sara R. Carlson, Neenah, WI (US); Reynee Wynne Kachur, Neenah, WI (US); Adam J. Meyer, Neenah, WI (US); Chris Rueb, Woodbury, MN (US); Robert Bowman, Woodbury, MN (US); Michael Lent, Woodbury, MN (US); William Hendrickson, Woodbury, MN (US); Jenna Foth, Hortonville, WI (US)

(73) Assignee: GPCP IP Holdings LLC, Atlanta, GA (US)

(21) Appl. No.: 17/637,884

(22) PCT Filed: Sep. 22, 2020
(86) PCT No.: PCT/US2020/051895

§ 371 Date: Feb. 24, 2022

Related U.S. Application Data
(60) Provisional application No. 62/904,942, filed on Sep. 24, 2019.

(51) Int. Cl. A61K 8/11; A61K 8/60; A61K 8/92; A61K 8/99; A61Q 19/00; A61Q 19/10; A61Q 5/02

(52) U.S. Cl. CPC A61K 8/11; A61K 8/60; A61K 8/92; A61K 8/922; A61K 8/99; A61Q 19/00; A61Q 19/10; A61Q 5/02; A61K 2800/84

(57) ABSTRACT

Microencapsulated freeze-dried probiotics along with methods of making and using them are disclosed. More specifically, the present disclosure relates to a probiotic microcapsule comprising a probiotic stasis pod, a nutrient rich carrier and a protective barrier layer. The microencapsulated probiotics as described are useful for inclusion in consumer and skincare products.

### CROSS-REFERENCE TO RELATED APPLICATIONS

This application claims priority to U.S. Provisional Patent Application No. 62/904,942, filed on Sep. 24, 2019, which is incorporated herein by reference in its entirety.

### BACKGROUND

### Field

The present disclosure relates to a microencapsulated probiotic. More particularly, the present disclosure relates to a probiotic microcapsule comprising a probiotic stasis pod, a nutrient rich carrier and a protective barrier layer. Still more particularly, the disclosure relates to a microencapsulated probiotic that can be incorporated into aqueous based compositions without compromising the probiotic. The present disclosure further relates to products containing the microencapsulated probiotic and methods of making and using the same.

Probiotics are live bacteria and yeast that are known to provide a variety of health benefits, particularly in the digestive system. The digestive system, being such a harsh environment, has spurred much research on ways to deliver probiotics to a host. Because probiotics are living cells, they must be protected if they are to remain viable until they reach the host that can receive the expected health benefit.

Probiotics have found many uses outside of the digestive system. Recently, it has been discovered that an imbalance in the human biome can be a substantial cause of conditions, including for example, skin irritations and eczema. Adding beneficial bacteria back to the skin through contact with probiotics has shown to improve skin barrier function, counteract inflammatory diseases such as eczema, and reduce the microbes that cause acne.

Currently many cosmetics, personal care products, and pharmaceuticals are marketed as containing live probiotics; however, what might have been formulated using live bacteria, do not end up retaining live bacteria. Modern cosmetics and pharmaceuticals must contain preservatives and other shelf stabilizing compositions that can kill the probiotic. Worse yet, even if the probiotic is protected and does remain viable until application, the contact between the probiotic and the surrounding carrier composition at the time of application can result in the same preservatives killing the probiotic before it can have the expected health benefits.

In addition to interfering with probiotic viability, the high level of preservatives that are required for adequate shelf-life of a cosmetic can also damage the human biome by killing naturally occurring microbes. The residual activity of preservatives found in products like skin care lotions can kill large numbers of beneficial bacteria that are naturally found on the skin. As a result, areas with a dearth of healthy bacteria, provide an opportunity for pathogens like Clostridium difficille, Methicillin Resistant Staphylococcus aureus (MRSA), or Vancomycin-Resistant Enterococci (VRE) to grow. Skin is believed to follow the lush lawn theory: if a lawn is lush and full, it is difficult for weeds to grow. If skin is full of healthy vibrant beneficial natural (flora) microbes, it is more difficult for pathogens to become established.

In some cases, the probiotic that is added to the cosmetic is in a lysed form. Lysed probiotics are those that have been chemically cleaved into many pieces and accordingly, they are not alive, so the issue of viability would seem less urgent. Without comment on whether or not there are benefits derived from bacteria parts, the cellular parts delivered to the host will likely also be compromised by the preservatives or other ingredients in the carrier cosmetic formulation.

Probiotic encapsulation has been contemplated for a number of years, to improve the survival of living probiotics in a range of formulations. Probiotic survival can be affected by a number of factors including, by way of example, pH and temperature. Encapsulation of the probiotic creates a physical barrier between the living probiotic and its surroundings, be they stomach acid, or pharmaceutical excipients. Current probiotic encapsulation technology (PET) includes encapsulation, entrapment and immobilization within a variety of biocompatible materials. While substantial research has been conducted on ways to maintain the viability of a living probiotic until it can reach the point of release and benefit, the currently available solutions remain wholly inadequate.

In co-owned prior application 62/648,553 filed Mar. 27, 2018, an improved microencapsulated probiotic is described for delivering live probiotic to a host. In the embodiment described, the live probiotic is microencapsulated with a polymer and nutrients which are then covered with a barrier coating. The embodiments disclosed in the prior application were stable in a variety of environments but were found particularly useful in low water environments.

The present disclosure provides a microencapsulated living probiotic that has an extended shelf life over the products described in the prior application and which can also be easily incorporated into aqueous systems without probiotic death making it possible to deliver living probiotics to the area of the host via an extended number of products that are in common use, for example, shampoos, skin lotions and other personal care products. The microencapsulation process as disclosed herein pretreats the probiotic before dispersion and encapsulation to further improve stability. The microencapsulated pretreatment vehicle described herein further protects the probiotic from the surrounding composition after the microcapsule is ruptured thereby improving the likelihood that the living probiotic properly contacts the intended area of the host.

### SUMMARY OF THE DISCLOSURE

The present disclosure relates to a microencapsulated probiotic including a core comprising a stasis pod containing a probiotic, a nutrient-rich carrier or a polymeric core and a protective layer surrounding the nutrient-rich carrier or polymeric core.

The present disclosure further relates to a probiotic containing microencapsule comprising one or more stasis pods comprising a freeze-dried probiotic and an adjuvant, a nutrient-rich carrier surrounding the stasis pods and a protective layer surrounding the nutrient-rich carrier.

In one embodiment, the present disclosure relates to microcapsules comprising at least one stasis pod comprising at least one freeze-dried probiotic and at least one adjuvant; a nutrient-rich carrier surrounding the at least one stasis pod; and a protective layer surrounding the nutrient-rich carrier.

According to another embodiment, the disclosure relates to a method for making an encapsulated probiotic comprising, ball-milling a freeze-dried probiotic in a water-saturated oil to a diameter of from about 2 μm to about 120 μm, mixing the freeze-dried probiotic and an adjuvant to form a stasis pod, surrounding the stasis pod with a nutrient-rich carrier; and encapsulating the nutrient-rich carrier and stasis pods in a protective coating.

According to yet another embodiment, the instant disclosure relates to skin and skin care products that comprise microencapsulated probiotics as described and claimed.

Additional advantages of the described methods and products will be set forth in part in the description which follows, and in part will be obvious from the description or may be learned by practice of the disclosure. The advantages of the disclosure will be realized and attained by means of the elements and combinations particularly pointed out in the appended claims.

It is to be understood that both the foregoing general description and the following detailed description are exemplary and explanatory only and are not restrictive of the invention, as claimed. The accompanying drawings, which are incorporated in and constitute a part of this specification, illustrate several embodiments and together with the description, serve to explain the principles of the disclosure.

### BRIEF DESCRIPTION OF THE DRAWINGS

FIG. 1 illustrates one embodiment of a microencapsulated probiotic according to the disclosure having a first stasis pod.

FIG. 2 illustrates one embodiment of a microencapsulated probiotic according to the disclosure having a second stasis pod.

FIG. 3 illustrates one embodiment of a microencapsulated probiotic according to the disclosure having a combination of stasis pods.

FIG. 4 illustrates one embodiment of a microencapsulated probiotic according to the disclosure having a combination of stasis pods and an enhanced nutrient-rich carrier.

FIG. 5 demonstrates probiotic survival in various carrier fluids.

FIG. 6 illustrates the stability of L. rhamnosus in various carriers over a six-week period.

FIG. 7 illustrates ball milling properties as a function of milling speed.

FIG. 8 illustrates particle size of freeze-dried L. rhamnosus as a function of time and relative speed.

### DESCRIPTION

Reference will now be made in detail to certain exemplary embodiments, examples of which are illustrated in the accompanying drawings. Wherever possible, the same reference numbers will be used throughout the drawings to refer to the same or like items.

The present disclosure relates to a probiotic microcapsule, a process for microencapsulating the probiotic, compositions containing the probiotic microcapsule, and uses and treatments using the probiotic microcapsule.

The probiotic microcapsule as described herein includes a probiotic material that is encased in a manner that prevents death of the live probiotic material before it can be delivered to a host in need of the probiotic. The microcapsule is made up of a number of components, each of which serves a different purpose in protecting the probiotic material. The microcapsule may include all or a subset of: a stasis pod containing a probiotic, a nutritive composition dispersed in the stasis pods or as a layer around the pods, an encapsulating layer that protects the core of probiotic and nutritive material, a moisture protective layer and a fugitive layer. The moisture protective layer and fugitive layers are optional and may or may not be used in combination with all of the embodiments.

As used herein, “encapsulation layer,” “encapsulation coating,” “encapsulation layers,” “shell,” and “encapsulate shell” are interchangeable and refer to the protective layer that surrounds the probiotic and nutritive composition.

In the description that follows, a layer or coating may be a single distinct coating or layer or may be made up of a number of layers. Unless indicated to the contrary, whether the term “layer” or “layers” is used, it should be understood that all embodiment can include either the plural or the singular.

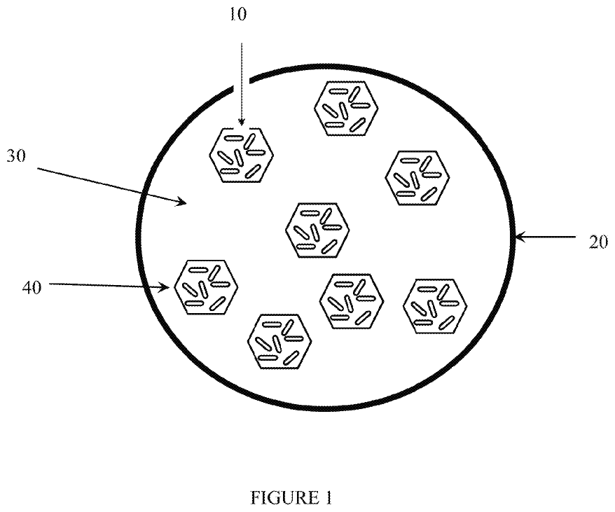

FIG. 1 illustrates a single probiotic microcapsule of the instant disclosure comprising a probiotic 10 that has been freeze-dried and combined with one or more adjuvants to form a stasis pod, 40. The pods 40 are surrounded by a nutrient-rich carrier 30 which is surrounded by a protective encapsulation coating 20. According to the embodiment seen in FIG. 1, the stasis pod 40 can include one or more probiotics each of which may be associated with its own carrier. Probiotic carriers can be nutritive or non-nutritive and are usually provided by the manufacturer of the probiotic material. The probiotic material is generally received from the manufacturer in a freeze-dried state. Freeze drying is a multi-step dehydration process in which a material is frozen and water is minimized by sublimation, leaving a dehydrated and preserved product. This preservation technique puts the probiotic materials used in the embodiments described into a state of stasis. In the event the desired probiotic material is not shipped in a freeze-dried state, the material may be freeze-dried using any art recognized technique prior to combination of the probiotic with an adjuvant to form the stasis pod 40.

The stasis pod 40 is the combination of one or more probiotics with an adjuvant, for example, a solid support such as silica, or wax. In the embodiment of FIG. 1, the stasis pods 40 are represented as all being the same, e.g., the pods 40 are all wax based or all silica based.

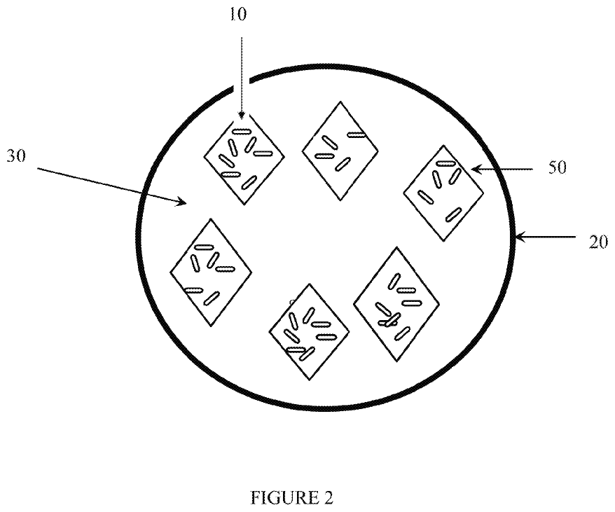

In the embodiment of FIG. 2, the microcapsule again contains a number of stasis pods, this time 50, indicating a probiotic 10 that has been freeze-dried and combined with one or more different adjuvants to form a different stasis pod 50. The pods 50 are again surrounded by a nutrient-rich carrier 30 which is surrounded by a protective encapsulation coating 20. As seen in FIG. 2, the stasis pods 50 may contain different amounts of probiotic material.

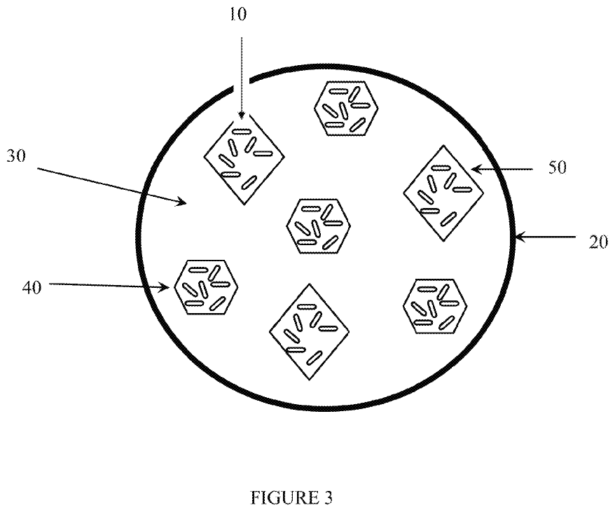

FIG. 3 illustrates another embodiment that includes stasis pods 40 and 50 which are different and which may include different probiotics and different adjuvants. The pods 40 and 50 are again surrounded by a nutrient-rich carrier 30 which is surrounded by a protective encapsulation coating 20.

In the commercial production of microcapsules of all embodiments, each stasis pod 40 or 50 will have its own unique amount of probiotic/carrier, and each microcapsule will have its own unique amount/number of stasis pods 40 or 50. Based upon the specific composition, the average relative particle size of both the stasis pods and the microcapsules, a range of the level of probiotic in the microcapsules can be calculated by the skilled artisan

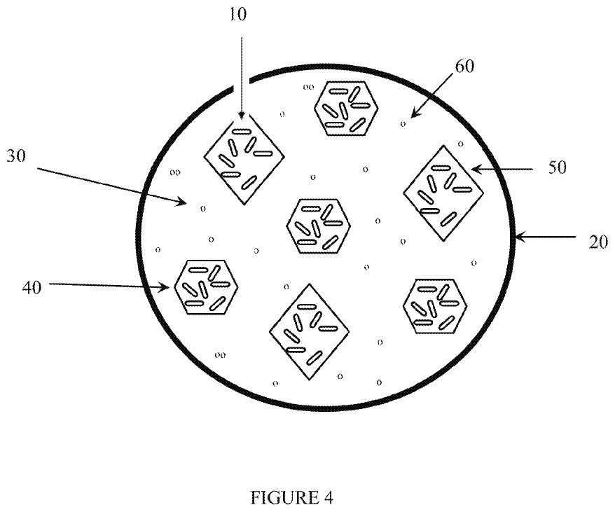

FIG. 4 addresses the same embodiment as seen in FIG. 3 further including a nutritive-rich carrier that includes additional nutritive particles 60, for example, cacao.

Probiotics for use in the instant disclosure can be chosen from any art recognized probiotic that one would want to protect until administration of the probiotic to the appropriate host. Such probiotics include one or more of Bifidobacterium, Pediococcus, Leuconostoc, Micrococcus, Escherichia, Staphylococcus, Streptococcus, Candida, Bacillus, and combinations thereof. Exemplary probiotics may include Staphylococcus, including S. epidermidis and S hominis; Propionibacterium including P. acnes, P. australiense, P. avidum, P. cyclohexanicum, P. granulosum, P. jensenii, P. microaerophilum, P. propionicum, P. thoenii, P. freudenreichii; Micrococci including M. antarcticus, M. luteus, M. lylae, M. roseus, M. agilis, M. kristinae, M. sedentarius, M. halobius; Cornebacterium including C. diphtheriae, C. efficiens, C. glutamicum; Malassezia (Yeast) including M. furfur, and combinations thereof. Preferred probiotics include those that can grow on, adhere to, or release beneficial proteins or DNA to the skin. Appropriate probiotics and prebiotics for use in the instant disclosure can be found, for example, in the Handbook or Probiotics and Prebiotics by Yuan Kun Lee and Seppo Alminen, second edition.

Some probiotics can be analogized to small biochemical manufacturing facilities where each microbe is a biochemical plant that keeps producing beneficial biochemicals. Microbes that naturally grow on the skin are those most often thought of in this way. According to one embodiment, the biochemicals of the probiotic can safely and naturally disinfect the skin by selective destruction of specific pathogens without substantial harm to the natural biome on the skin. According to another embodiment, the biochemicals can reduce the inflammation response and reduce or eliminate eczema or skin irritation. According to yet another embodiment, the biochemicals of the probiotics can strengthen the skin and induce natural ceramide production—which is to say that skin is rejuvenated, made younger, and made more resistant to the effects of pollution and aging.

Other probiotics can be understood as delivery vehicles that drop off the beneficial ingredients and then move on to deliver the ingredients somewhere else. These are generally the microbes that cannot grow on the skin. Instead they release beneficial peptides and DNA delivering unique benefits to the skin. The DNA and peptides carried by these probiotics are proteins, and as such, they interact with the chemistry of the surrounding delivery vehicle or composition base. Specifically, proteins cannot function properly when they chemically attach to cationic ingredients or become misshapen due to acids or bases. The only way to successfully deliver these proteins to a host is to deliver the proteins in a viable bacteria that naturally breaks when exposed to air. These probiotic bacteria (e.g. anaerobic bacteria) can be likened to a microcapsule that is, according to the described invention, then microencapsulated within another microcapsule. According to one embodiment, anaerobic bacteria delivered to the skin may enter the pores and survive naturally, delivering the beneficial proteins to the host.

According to one embodiment, the encapsulated microbes may be chosen from those that can produce peptides that kill pathogenic bacteria. When such a product is applied to the skin, it provides anti-bacterial properties that may extend beyond the point of first application.

Below Tables 1 provides a description of probiotic materials that can be used in the processes described herein. Many are preferred for use on the skin.

TABLE 1
Beneficial Skin Microorganisms

| | | | | |
|---|---|---|---|---|
| | Colony | | Spore | |
| | Morphology | | Forming/ | |
| | (Gram-Positive | Oxygen | Non-Spore | |
| Name | vs. Gram-Negative) | Requirements | Forming | Comments |

Staphylococcus

| | | | | |
|---|---|---|---|---|
| S. epidermidis | Gram-positive | Aerobic | Non-Spore | Lives on the skin and |
| S. hominis | | | Forming | nostrils of all humans. |

Propionibacterium

| | | | | |
|---|---|---|---|---|
| P. acnes | Gram-positive | Anaerobic | Non-Spore | Generally nonpathogenic. |
| P. australiense | | | Forming | Commonly found on |
| P. avidum | | | | the skin (mostly on |
| P. cyclohexanicum | | | | face and surrounding |
| P. granulosum | | | | areas) in numbers |
| P. jensenii | | | | ranging from <10/cm2 |
| P. microaerophilum | | | | to 106/cm2. |
| P. propionicum | | | | |
| P. thoenii | | | | |
| P. freudenreichii | | | | |

Micrococci

| | | | | |
|---|---|---|---|---|
| M. antarcticus | Gram-positive | Aerobic¹ | Non-Spore | Commonly found on the skin. |
| M. luteus | | | Forming | Grow well in |
| M. lylae | | | | environments with |
| M. roseus | | | | little water and high |
| M. agilis | | | | salt concentrations. |
| M. kristinae | | | | |
| M. sedentarius | | | | |
| M. halobius | | | | |

Corynebacterium

| | | | | |
|---|---|---|---|---|
| C. diphtheriae | Gram-positive | Facultative | Non-Spore | Found in human |
| C. efficiens | | Anaerobic | Forming | mucous membranes |
| C. glutamicum | | | | and on skin. |

Malassezia (Yeast)

| | | | | |
|---|---|---|---|---|
| M. furfur | Gram-Positive | Facultative | Non-Spore | One of the only |
| | | Anaerobic | Forming | species of fungi that |
| | | | | are a part of the |
| | | | | normal human skin flora. |
| | | | | Found on the chest, |
| | | | | shoulders, and arms. |
| | | | | More commonly found |
| | | | | on Caucasian |
| | | | | individuals and adults |
| | | | | over the age of 12. |

Below, Table 2 lists probiotics that are not harmful to skin, but that create peptides that target non-native bacteria (pathogens). Besides targeting C. difficile, MRSA, and VRE, probiotics can be used to target other pathogens of concern are listed below:

TABLE 2
Pathogenic Skin Microorganisms.

| | | | | |
|---|---|---|---|---|
| | Colony | | Spore | |
| | Morphology | | Forming/ | |
| | (Gram-Positive vs. | Oxygen | Non-Spore | |
| Name | Gram-Negative) | Requirements | Forming | Comments |

Escherichia coli

| | | | | |
|---|---|---|---|---|
| E. coli | Gram-negative | Facultative | Non-Spore | More commonly found in |
| | | Anaerobic² | Forming | intestine, but are frequently |
| | | | | isolated from skin and soft |
| | | | | tissue infections (surgical/ |
| | | | | traumatic wounds). |

Pseudomonas

| | | | | |
|---|---|---|---|---|
| P. aeruginosa | Gram-negative | Aerobic/ | Non-Spore | Opportunistic human |
| | | Facultative | Forming | pathogen. Typically |
| | | Anaerobic | | colonize in |
| | | | | immunocompromised |
| | | | | humans. |
| | | | | Leading Gram-negative |
| | | | | opportunistic pathogen in |
| | | | | most medical centers (40- |
| | | | | 60% mortality rate). |

Enterococci

| | | | | |
|---|---|---|---|---|
| Vancomycin- | Gram-Positive | Facultative | Non-Spore | Serious infections usually |
| Resistant | | Anaerobic | Forming | occur in hospitalized |
| Enterococcus | | | | patients |
| (VRE) | | | | |

Staphylococcus

| | | | | |
|---|---|---|---|---|
| S. aureus | Gram-positive | Facultative | Non-Spore | Typically spread from |
| S. aureus | | Anaerobic | Forming | human to human via hands. |
| (MRSA) | | | | S. aureus colonizes |
| S. aureus | | | | naturally in the nose, |
| (VRSA) | | | | mouth, mammary glands, hair, |
| | | | | upper respiratory tract, etc. |

Candida (Yeast)

| | | | | |
|---|---|---|---|---|
| Candida | Gram-Positive | Facultative | Yeast spores | Found primarily in the |
| albicans | | Anaerobic | form on | intestines, colon, and mouth. |
| | | | chlamydospores. | Mostly attacks skin or |
| | | | | mucous membranes. |
| | | | | Other infections may |
| | | | | include areas such as the |
| | | | | armpits, groin, and skin folds. |

Clostridium

| | | | | |
|---|---|---|---|---|
| C. tetani | Gram-Positive | Anaerobic | Spore Forming | Known to produce a |
| C. botulinum | | | | variety of toxins (some of |
| C. perfringens | | | | which are fatal). |
| C. acetobutylicum, | | | | Spores are passed in |
| C. difficile | | | | feces and spread to food, |
| C. novyi | | | | surfaces, and objects |
| | | | | through individuals who do |
| | | | | not wash hands properly. |
| | | | | Spores may persist up to months. |

Freeze-dried probiotics are generally produced with one or more carrier materials for stabilization and transportation. Prebiotic sugars, for example, manose, mannitol, maltodextrin, lactulose, trehalose, and sorbitol are typical carrier materials for probiotics. If the probiotic is acquired without a carrier, it may be included in a stasis pod 40 without a carrier or after the addition of a carrier. Alternatively, if the probiotic is acquired with a carrier, the carrier may be removed or retained prior to dispersion of the probiotic material into the stasis pod 40.

If the freeze-dried probiotic includes a carrier or a carrier is desired and added, the probiotic/carrier combination is ordinarily ball milled to generate probiotic/carrier particles having a diameter in the range of about 2 microns to about 120 microns, for example, from about 2 microns to about 110 microns, for example from about 20 to about 120 microns, for example from about 30 microns to about 120 microns prior to incorporation into the stasis pods. If the freeze-dried probiotic has no carrier or no carrier is desired, the probiotic can be incorporated without ball milling into the stasis pod.

According to another embodiment, the particle size of about 2 microns to about 120 microns can be reported as a D50, i.e., the maximum particle diameter below which 50% of the sample volume exists. According to yet another embodiment, the particle size range of about 2 microns to about 120 microns can be reported as a D90, i.e., the maximum particle diameter below which 90% of the sample volume exists. When particle diameter is reported as a D-50 or a D-90 as used herein, it will be so noted.

The freeze-dried probiotic material, with or without the carrier, is combined with one or more adjuvants to form the stasis pod. The adjuvants help to protect the probiotics during shelf life and also help to disperse the probiotics after the microcapsules protective shell 20 is broken during application. Adjuvants for use in the stasis pods include waxes and solid support carriers.

Waxes for use in the stasis pod may be chosen from one or more of organic esters and waxy compounds derived from animal, vegetable, and mineral sources including modifications of such compounds in addition to synthetically produced materials having similar properties. Specific examples that may be used alone or in combination include glyceryl tristearate, glyceryl distearate, vegetable oils such as canola wax, hydrogenated cottonseed oil, hydrogenated soybean oil, castor wax, rapeseed wax, beeswax, carnauba wax, candelilla wax, microwax, polyethylene, polypropylene, epoxies, long chain alcohols, long chain esters, long chain fatty acids such as stearic acid and behenic acid, hydrogenated plant, and animal oils such as fish oil, tallow oil, and soy oil, microcrystalline waxes, metal stearates, white grease, yellow grease, and brown grease, and metal fatty acids. According to one embodiment, hydrophobic wax materials include for use in the instant disclosure include Dynasan™ 110, 114, 116, and 118 (commercially available from DynaScan Technology Inc., Irvine, Calif.), Sterotex™ (commercially available from ABITEC Corp., Janesville, Wis. Specific waxes, including edible waxes for use in the disclosed embodiments include gulf wax, beef tallow, mutton tallow, butter, vegetable shortening, cocoa butter, coconut oil and shea butter.

Solid support carriers for use in the stasis pod may be chosen from any supporting material that will not harm the probiotic material. Suitable supports may be chosen from one or more of maltodextrin; proteins, for example, from pea, soy, rice, hemp; starches such as potato, wheat, tapioca, corn, and rice; woven and non-woven fabric and pads; carbon, such as carbon black or activated carbon; metal oxides or any refractory oxides such as alumina, zirconia, magnesia, titania; silica, amorphous or crystalline, for example, fumed silicas, silica gels, precipitated silicas, precipitated silica gels, silicalite or mixtures; kieselguhr and other diatomaceous earths; silicates, for example, magnesium silicate and calcium silicate and mixtures, aluminosilicates including clays and zeolites; metal phosphate such as zirconium phosphate or mixtures or any of the above.

According to one embodiment, the stasis pods may also contain other water-soluble resources or one or more prebiotic sugars. Water soluble resources include, for example, proteins, nucleotides and ions such as sodium, potassium and calcium. Prebiotic sugars may be chosen from one or more of (e.g. dried (glucose corn syrup, fructose, manose, mannitol, maltodextrin, lactulose, treehalos, and sorbitol) oligosaccharides (e.g. Fructo-oliosaccharides—Raftilose P95 Orafti, Belgium), galactooligosaccharises, resistant starch-rich whole grains (e.g. oat β-glucan, flaxseed gum, fenugreek gum, and matured gum Arabic), and mannan oligosaccharide-rich yeast cell wall material is demonstrated to be a valuable prebiotic, and certain proteins (e.g. lactoferrin), certain plant extracts (e.g. luteins and black current extract powered). Prebiotic sugars or sugar alcohols act as a nutritive composition for the probiotics since they include compounds such as glucose, fructose, oligosaccharides, mannose, glucomannans hydrolyzate, xylitol, erythitol, or sorbitol, all of which encourage the growth of the probiotic microbes.

The stasis pod may be suspended in a nutrient-rich composition to form the composite 40 making up the core of the microcapsule. Nutrient-rich composition may be chosen from one or more of cocoa butter, coconut oil, flaxseed oil, shea butter, low melting fats, vegetable oils, silicone oil, mineral oils and the like.

According to one embodiment, the nutrient-rich composition may be an organogel made up of both a wax and an oil as described above. Examples of suitable organogels include for example, a combination of gulf wax and cyclopentasiloxane oil or a combination of gulf wax and mineral oil. After reviewing the information provided herein, appropriate combinations of wax and oil will be readily apparent to the skilled artisan.

Alternatively, the stasis pods may be suspended in a polymer to form a composite making up the core of the microcapsule. Any art recognized polymer(s) or combination of polymers useful for pharmaceutical applications can be used for suspending the freeze-dried probiotic including, but not limited to, polyethylene glycols (PEGs), polyvinyl pyrrolidone ((PVPs)—preferably with molecular weights between about 40,000 to about 360,0000), polyvinyl alcohol (PVAs), polyacrylamides, N-(2-hydroxypropyl) methacrylamide (HPMA), xanthan gum, guar gum, pectins, dextran, carrageenan, sodium carboxyethyl cellulose, polyacrylic acid polymers, hyaluronic acid, carboxyvinyl polymers, hydroxyethyl cellulose, cellulose, hydroxypropylmethyl cellulose, carboxyvinyl polymer. According to one embodiment, polyacrylic acid polymers, for example, Carbopol® Ultrez 20, Carbopol® Ultrez 21, both from Lubrizol, as well as HivisWako® a carboxyl vinyl polymer, from Wako Chemicals Ltd, can be used to suspend the freeze-dried probiotic.

The nutrient-rich carrier of FIG. 1 or the polymer core described above further includes a protective encapsulation coating 20. The encapsulation coating 20 may be from 1 to 30 layers thick, for example, from about 1 to about 20 layers thick, for example, from about 1 to about 10 layers thick. The coating weight of the encapsulation coating 20 may be from about 1% to about 50% of the microcapsule weight, for example, from about 1% to about 40% of the microcapsule weight, for example, from about 1% to about 30% of the microcapsule weight, for example, from about 1% to about 20% of the microcapsule weight, for example, less than 15% of the microcapsule weight.

According to one embodiment, the probiotic nutrient-rich carrier or polymeric core may be further coated with an oil layer before being subjected to encapsulation. The oil layer may provide insulation between the probiotic core and the encapsulation layer allowing higher temperature materials to be used during the microencapsulation process.

Suitable temperatures for the encapsulation coating 20 generally include 140° F., for example, less than about 100° F., for example, less than about 90° F., to facilitate the coating of the freeze-dried probiotic slurry. The encapsulation coating layer 20 may be comprised of a polymeric material, a crosslinked polymeric material, a metal, such as Ca²⁺, a ceramic or a combination thereof, that results in a shell material that may be formed during manufacturing. Specifically, the encapsulation coating layer may be comprised of crosslinked sodium alginate, anionic dispersed latex emulsions, crosslinked polyacrylic acid, crosslinked polyvinyl alcohol, crosslinked polyvinyl acetate, silicates, carbonates, sulfates, phosphates, borates, polyvinyl pyrollidone, PLA/PGA, thermionic gels, urea formaldehyde, melamine formaldehyde, polymelamine, crosslinked starch, nylon, ureas, including polymethylene urea, hydrocolloids, and combinations thereof. According to one embodiment, the crosslinked polymeric system is crosslinked sodium alginate.

According to another embodiment, the protective coating may be formed from a drying oil or shellac. Drying oils are oils that harden to a tough solid film after exposure to air for a period of time. Drying oils typically have unsaturated fatty acids, for example, linoleic or linolenic acids, glycerides, or carboxylic acids and harden based upon polymerization reactions with oxygen contained in the air. Drying oil may be chosen from one or more of linseed oil, Tung oil, poppy seed oil, perilla oil, walnut oil, soybean oil, tall oil, caster oil, and the like.

According to another embodiment, as an alternative to drying oils, shellac may be dissolved in ethanol and water and applied to the microcapsules after which the solvent is allowed to evaporate to form the protective coating layer.

The encapsulation coating layer generally has a thickness of from about 0.1 micrometers to about 500 micrometers, for example, from about from about 1 micrometer to about 100 micrometers, for example, from about 1 micrometer to about 50 micrometers, for example, from about 1 micrometer to about 20 micrometers, for example, from about 2 micrometers to about 10 micrometers. Suitable methods for measuring the thickness of the encapsulation layer 20 (once fractured), and the other optional layers described herein, include Scanning Electron Microscopy (SEM) and Optical Microscopy.

According to one embodiment, the encapsulation coating layer 20 is a single discrete layer. According to another embodiment, the encapsulation coating 20 comprises multiple layers added in one or more steps.

According to some embodiments, a moisture protective layer (not shown) may also be included. The moisture protective layer generally surrounds the encapsulation coating 20. The moisture protective layer can comprise one or more of the following compositions, alone or in combination. The materials are chosen from polyols in combination with isocynate, styrene-acrylate, vinyl tolueneacrylate, styrene-butadiene, vinyl-acrylate, polyvinylbutyral, polyvinyl acetate, polyethylene terephthalate, polypropylene, polystyrene, polymethyl methacrylate, polylactic acid, polyvinylidene chloride, polyvinyldichloride, polyethylene, alkyd polyester, carnauba wax, hydrogenated plant oils, hydrogenated animal oils, fumed silica, silicon waxes, titanium dioxide, silicon dioxide, metals, metal carbonates, metal sulfates, ceramics, metal phosphates, and microcrystalline waxes. When the moisture protective layer is used, it may be from about 5% to about 35% of the microcapsule weight, for example, from about 5% to about 30% of the capsule weight, for example, from about 5% to about 20%, for example, from about 5% to about 15%.

According to another embodiment, one or more fugitive layers (not shown) may be added to protect the microcapsule from process damage. The fugitive layer may be comprised of any one of several suitable materials including polylactic acid, polymers of dextrose, hydrocolloids, alginate, zein, and combinations thereof. According to one embodiment, the fugitive layer is starch. The fugitive layer protects the microcapsule during production. The layer may be applied to any of the layers of the microcapsule. The fugitive layer may be something that is eliminated during processing or something that may remain as part of the end product.

The probiotic microcapsules of the instant disclosure can be produced using any art recognized methods. According to one embodiment, the first step in the production of the encapsulated probiotic 10 is to select and freeze dry the probiotic materials or to obtain freeze-dried probiotic from a manufacturer.

According to one embodiment, the probiotic is selected from a combination of bacteria and/or yeast. To decrease production losses, the probiotic can be cooled to a temperature of 5° C. to 15° C. and dried by sublimation of the water from the organism. In some embodiments, the probiotic may be added to an excipient before being cooled. Such an excipient may contain an oil and/or a prebiotic sugar. Probiotics can be freeze-dried using any art recognized process. According to one embodiment, the probiotic powder is produced using standard freeze drying, spray drying, or chemical drying.

The freeze-dried probiotic/carrier combination can be ball milled to particle diameter D50 or D90 of from about 2 microns to about 120 microns using any art recognized ball milling equipment. One appropriate configuration is discussed in Example 3, below. Ball milling is preferably carried out in one or more oil based mediums, for example, one or more silicone, canola, mineral and cyclopentasiloane oils. According to one embodiment, the oil medium has been saturated with water.

After the probiotic particles have been sized, the freeze-dried probiotic slurry can be blended with an adjuvant to form a stasis pod.

In one embodiment, the freeze-dried probiotic slurry can be hydrophilic and would form droplets inside the wax at an elevated temperature. The selected temperature should be sufficient to melt the hydrophobic wax material, but maintained for a sufficiently short time to keep the freeze-dried probiotic viable. Temperatures can range from 80° F. to about 150° F. To maintain the viability of the freeze-dried probiotics, the temperature should not be maintained at or above 140° F., for longer than required to create the desired thickness of microcapsule. Appropriate times are based upon the specific materials being used and would be well understood by the skilled artisan.

According to another embodiment, the wax and probiotic particles may be subject to melt-spraying or prilling. In this embodiment, the molten wax was mixed with the probiotic and the combination is fed through a nozzle via a pumping action of by air pressure. From the nozzle, small droplets are formed which solidify upon cooling in passage through the air between the nozzle and a collection bath. Prilling is the process by which something congeals or freezes in mid-air after being dripped (or in our case, atomized). According to one embodiment, the wax containing the probiotic may be combined with another wax or oil that contains the water-soluble resources needed by the probiotic. In this embodiment, the first wax including the probiotic is combined with the second wax or oil and the two together are forced from the nozzle and caused to prill.

According to another embodiment, the probiotic material is combined with the solid support carrier. The probiotic material may be adhered to the solid support carrier using any art recognized method that is appropriate to the chosen probiotic and carrier. For example, silica may be impregnated with a probiotic material by contacting the silica and probiotic in cyclopentasiloxane (CPS) and heating to drive off the CPS. In this example, the silica may be impregnated over several steps until the desired loading is reached. Silica is able to hold a great deal of probiotic material per gram. Regarding other solid support carriers any form of physical or chemical attachment that doesn't harm the probiotic would be appropriate.

In one embodiment, if silica is the solid support carrier, the silica may be calcined to remove surface water prior to combination of the silica with the probiotic. The probiotic releases easily from the silica when it comes into contact with water.

According to another embodiment, the wax based stasis pods and the solid support carrier stasis pods can be mixed together before being encased in a nutrient-rich carrier or combined in a polymeric core material.

According to yet another embodiment one or more added nutrients may be incorporated into the nutrient-rich carrier before it is combined with the stasis pods and encapsulated. The added nutrients may include one or more of cacao, trehalose, mannose, dextrose, polysaccharides; prebiotics such as butyrate, acetate and propionate; glucomannan or chicory root.

The stasis pods of freeze-dried probiotic are next encased in a nutrient-rich carrier or polymeric material to form the core of the microcapsule. In one embodiment when a nutrient-rich carrier is used, the carrier is heated to increase moldability and combined with the stasis pods. The carrier surrounds the pods creating a solution that may be particlized using any art recognized process.

According to one embodiment, when a polymer is used, the polymer is dissolved in water at a high enough concentration to suspend the freeze-dried probiotic stasis pods, but at a low enough concentration to allow the solution to flow through the microencapsulation process equipment. Polymer concentrations are generally between 5% and 60%, for example, from about 40% to about 60%, when using a sugar/protein or wax matrix, and for example, from about 5% to about 15% when using a polymeric matrix.

According to another embodiment, the microcapsule may be formed by including an activator or crosslinker in the nutrient-rich carrier or polymer core. The encapsulating activator may be any activator capable of initiating a crosslinking reaction in the presence of a crosslinkable compound. Suitable encapsulating activators include polyvalent ions of calcium, polyvalent ions of copper, polyvalent ions of barium, silanes, aluminum, titanates, chelators, acids, or combinations of these. According to one embodiment, the activator is calcium chloride or calcium combined with any number of anions.

According to this embodiment, encapsulating the freeze-dried probiotic/polymer slurry in the presence of an activator in the core composition allows for almost instantaneous crosslinking when the core composition is introduced into the solution containing the crosslinkable compound. This immediate crosslinking reduces the potential for unwanted freeze-dried probiotic hydration. According to one embodiment, the freeze-dried probiotic/polymer slurry may be added dropwise into the liquid containing the crosslinkable compound and the beads that form when the drops contact the liquid will form an encapsulating coating. Stirring can provide sufficient disruption to maintain the individual beads separate during the crosslinking reaction. Agglomerated masses can be susceptible to numerous defects and while they may be physically separated, it is preferable that they not be formed. Any art recognized method, physical or chemical, for achieving bead separation may be used. The drops added to the liquid solution may have a diameter of from about 0.05 millimeters to about 1 millimeter, for example from about 0.1 millimeters to about 1 millimeter.

When the core composition including the encapsulating activator is introduced into the liquid containing the crosslinkable compound, the encapsulating activator migrates to the interface between the core composition and the liquid solution and initiates the crosslinking reaction on the surface of the core composition to allow the encapsulation layer to grow outward toward the liquid solution.

The thickness of the resulting encapsulation layer surrounding the core composition can be controlled by (1) controlling the amount of encapsulating activator included in the core composition; (2) controlling the amount of time the core composition including the encapsulating activator is exposed to the liquid solution including the crosslinkable compound; and/or (3) controlling the amount of crosslinkable compound in the liquid solution. By way of example, a solution including alginate in a range of from about 1 to about 500 mg/ml, CaCl₂) in a range of from about 0.1 to about 100 mg/ml level and at a temperature between about 4° C. and about 37° C., would produce a thickness of between 1-20 μm of alginate.

According to another embodiment, the core composition may be introduced or poured into a liquid solution including the crosslinkable compound and then subjected to shear sufficient to break the paste into small beads for crosslinking. Any art recognized method of applying the shear may be used.

According to one embodiment, the liquid solution includes a crosslinkable compound that can be crosslinked in the presence of the encapsulating activator and a surfactant to form the outer encapsulate shell. The surfactant can be chosen from one or more sugar or sugar-based surfactants, e.g., Tween 20, or amino acid or protein-based materials

According to another embodiment, the encapsulation coating 20 may can be formed using a process known as coacervation, which may not require a chemical encapsulating activator to be present in the core composition. Coacervation processes can utilize a change in pH, a change in temperature, and/or a change in ionic strength of the liquid solution to initiate the formation of the encapsulating layer around the core composition.

Although it is generally desirable to locate the encapsulating activator in the core, according to one embodiment, the encapsulating activator may be in the liquid solution. In this embodiment, the encapsulating activator chemically reacts with the crosslinkable compound also contained in the liquid solution. The resulting microencapsulated freeze-dried probiotic slurry may be free from any encapsulating activator or it may contain a small amount of encapsulating activator not consumed in the crosslinking reaction.

According to some embodiments, microencapsulated freeze-dried probiotics can be subjected to a process to impart a moisture protective layer that surrounds the encapsulated layer that comprises the crosslinked compound. This moisture protective layer can for appropriate products provide the microencapsulated freeze-dried probiotic with increased protection from water; that is, it can make the microencapsulated freeze-dried probiotic substantially fluid impervious and allow the microencapsulated freeze-dried probiotic to survive long term in an aqueous environment and not degrade until the moisture protective layer is ruptured by mechanical action. The moisture protective layer may be a single layer applied onto the microencapsulated freeze-dried probiotic, or may comprise several layers one on top of the other.

The moisture protective layer may be applied to the microencapsulated freeze-dried probiotic utilizing any number of suitable processes including, atomizing, or dripping a moisture protective material onto the microencapsulated freeze-dried probiotic. Additionally, a Wurster coating process may be utilized. When a solution is used to provide the moisture protective coating, the solids content of the solution is generally from about 5% to about 40%, for example, from about 5% to about 30%, for example from about 5% to about 20%, for example from about 10% to about 20%. Generally, the viscosity of the solution is from about 20 cp to about 500 cp, for example from about 20 cp to about 80 cp, for example, from about 30 cp to about 70 cp.

According to one embodiment, a fluidized bed process can be utilized to impart the moisture protective layer on the microencapsulated freeze-dried probiotic. The fluidized bed is a bed or layer of microencapsulated freeze-dried probiotic through which a stream of heated or unheated carrier gas is passed at a rate sufficient to set the microencapsulated freeze-dried probiotic in motion and cause them to act like a fluid. as the microcapsules are fluidized, a spray of a solution comprising a carrier solvent and the moisture protective material is injected into the bed and contacts the vehicles imparting the moisture protective material to the outside of the microcapsule. The treated microcapsules are collected when the desired moisture protective layer thickness is achieved. The microencapsulated freeze-dried probiotic can be subjected to one or more fluidized bed processes to impart the desired level of moisture protective layer.

According to some embodiments, the microencapsulated freeze-dried probiotic, can be subjected to a process for imparting a fugitive layer surrounding the outermost layer. The fugitive layer could be applied on the freeze-dried probiotic such that it substantially completely covered either the nutrient-rich carrier, core or moisture protective layer. The fugitive layer may be applied to the microencapsulated freeze-dried probiotic utilizing any number of suitable processes including, atomizing, or dripping a fugitive material onto the microencapsulated freeze-dried probiotic. When a solution is used to provide the fugitive coating, the solids content of the solution is from about 10% to about 60%, for example, from about 10% to about 50%, for example, from about 20% to about 50%. The pH of the solution is from about 2.5 to about 11. The viscosity of the solution may be from about 20 cp to about 100 cp, for example from about 20 cp to about 80 cp, for example, from about 30 cp to about 70 cp. The preferred method of applying the fugitive layer utilizes a fluidized bed reactor. Alternatively, any art recognized coating process may be used, including a Wurster coating process.

These probiotic microcapsules can be used in a vast number of ways and in a variety of compositions. For example, they can be used to 1) deliver living probiotic microbes to the skin to improve skin barrier function, 2) deliver living probiotic microbes to the skin to reduce inflammation associated with acne, eczema, rosacea, or contact dermatitis, 3) strengthen the human biome so pathogenic bacteria do not have a chance to colonize, 4) deposit probiotics on skin that produce anti-pathogenic peptides which kill pathogens; 5) improve the skins ability to ward off pathogen colonization near medical devices such as insulin pumps or catheters; 6) reduce or eliminate the need for preservatives in cosmetic products; 7) reduce skin ulcers on bedridden, diabetic, or otherwise compromised individuals, 8) protects skin from and fights cancer.

Micro-encapsulated probiotics can be used in compositions including, for example, moisturizing lotion, sunscreen, lip balm, oral care product, shampoo, soap, hair conditioner, baby wipes, perineal wipes, facial cleaning wipes, feminine hygiene pad or tampon, diaper or adult incontinence product, deodorant (roll-on liquid, spray, or stick), pet food, food additives, food condiments, medicinal products, bandages and the like.

In use, the microcapsules are broken by physical forces that are applied to the capsules. Accordingly, depending upon the end use, the size and thickness of the microcapsules can play a big role in the release of the probiotics. Thinner capsule walls would generally result in more delicate capsules.

After understanding the information disclosed herein, the production of other carrier compounds suitable for use with the described microencapsulated product would be readily understood by the skilled artisan.

According to one embodiment, the carrier composition, for example, a lotion, sunscreen, deodorant, could be irradiated to assure it was free from microbial contamination. The irradiated product could be blended with freeze-dried probiotic. The product could them be packaged in an appropriate OTR material and sold. The freeze-dried probiotic should stay in stasis until the time of use of the product.

According to one embodiment, oxygen transmissive packaging may allow the microencapsulated probiotics to remain viable without refrigeration. Containers for the products described herein may be any standard art recognized packaging. Packaging compositions with the described microencapsulated probiotic in packages with low oxygen transmission may reduce shelf-life, but would not otherwise interfere with the product.

According to one embodiment, compositions comprising the microencapsulated probiotic(s) are packaged in high oxygen transmission packaging which can improve both shelf-like and/or bacterial viability without refrigeration. Oxygen transmission rate (OTR) is well understood and high OTR packaging is used for products that require substantial oxygen concentrations, such as contact lenses. See, for example, U.S. Pat. No. 9,062,180. However, current technology surrounding cosmetic products is to provide a good oxygen barrier. See, for example, U.S. Pat. No. 8,124,204. Probiotics benefit from high oxygen environments and for that reason their packaging should be produced from high OTR materials. Like the prebiotic sugar, high oxygen may extend the life of microbes without refrigeration thereby creating a shelf-stable product at room temperature. Further, carrier compositions that have very low-water-activity will have much less degradation based upon exposure to oxygen.

According to one embodiment, the probiotic container could be a single layer of any number of materials including:

TABLE 3

| Material | Oxygen Transmission Rate |
|---|---|
| High Density Polyethylene (HDPE), | OTR = 150-200 |
| Low Density Polyethylene (LDPE) | OTR = 450-500; |
| Includes Linear Low-Density | |
| Polyethylene (LLDPE), | |
| Polyethylene Terephthalate (PET, | OTR = 280-400 |
| PETE), | |
| Polypropylene (PP), | OTR = 100-160 |
| Polystyrene (PS), | OTR = 280-400 |
| Polyvinyl Chloride (PVC, Vinyl) | OTR = 0.35-0.50 |
| Polylactic Acid (PLA) | OTR = 38-42 |
| Polycarbonate (PC) | OTR = 160 @ 24 hr. |
| Polytetrafluoroethylene or Teflon ® | OTR = 17 @ 24 hr. |
| (PTFE) | |
| Silicone | Range of OTR depending on type. |

Oxygen requirements for various microbes can be found in the literature. According to one embodiment, for oxygen loving microbes, LDPE packaging could be used. For microbes that respond better with lower oxygen conditions, the packaging could be selected with lower OTR per the list above. For all packaging, the oxygen transmission should not be so high that the probiotic leaves stasis.

The methods and products described herein should not be limited to the examples provided. Rather, the examples are only representative in nature.

### EXAMPLES

### Example 1

Stabilized probiotics including L. rhamnosus, L. paracasei, L. salivarius and S. thermophilis were obtained from the manufacturer stabilized in a network of trehalose, a prebiotic carrier. The freeze-dried probiotic as received was dispersed in an oil carrier and was then balled milled. The probiotic in the trehalose are approximately about 0.5 to about 2.5 microns in size.

Oil-based carriers, ball milling conditions and particle diameters were varied. The carrier, ball milling conditions and particle size all effect the survival rate of the probiotic as will be shown below. However, once the probiotic particles have stabilized in oil (after the end of about one week), the bacteria counts remain shelf stable for an extended period of time.

Each of the bacteria were incorporated into four different oils including silicone, canola, mineral and cyclopentasiloane. All except the L rhamnosus were also tested in coconut oil. The oils were saturated with water according to the following Table.

| | | Water | | Water |
|---|---|---|---|---|
| | | KF | Water | KF |
| Oil | Fresh | (%) | add | (%) |
| Corn | X | 0.01 | Saturated | 0.11 |
| Canola | X | 0.01 | Saturated | 0.09 |
| Mineral | X | 0.00 | Saturated | 0.00 |
| Decamethylcyclopentasiloxane | X | 0.00 | Saturated | 0.00 |
| Silicone oil 350 cSt | X | 0.00 | Saturated | 0.01 |
| Glycerin | X | 0.03 | 2% | 2.07 |
| Triethyl citrate | X | 0.05 | 2% | 1.95 |

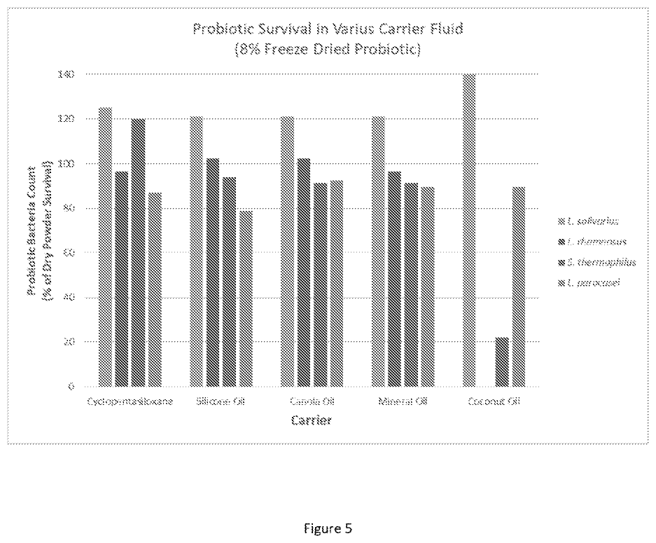

The results of these tests can be seen in FIG. 5. As seen in FIG. 35 L. salivarius survived and thrived in all of the oil-based carriers. L rhamnosus was also very stable in all carriers in which it was tested. S. thermophilis was stable in all except coconut oil. Finally, L. paracasei survived in each of the oils at a rate of between about 78 and 90%. As is clear from these results, the probiotics can be dispersed in different oils depending upon the specific probiotic or probiotic mixture. The results also show that cyclopentasiloxane may be a preferred carrier for use in the products as described in the instant specification.

### Example 2

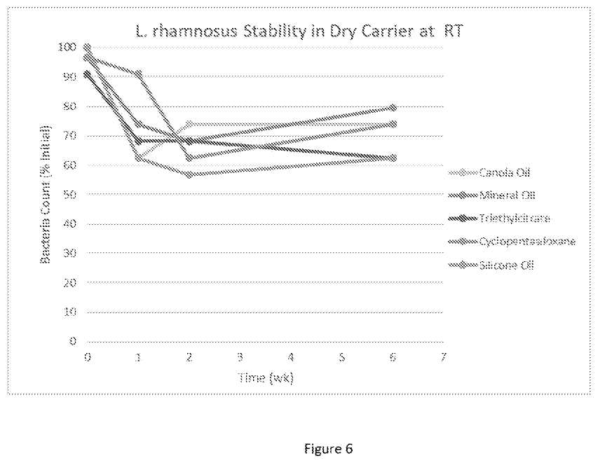

The stability of L. rhamnosus was tested in each of the carriers to determine how well the bacteria would survive over 6 and 12 weeks. As seen in FIG. 6, the L. rhamnosus survived at a rate of 60% to 80% at room temperature depending upon the carrier over a six week period. While the bacteria count in silicone oil had dropped by nearly 40%, the count in canola oil, cyclopentane and mineral oil were all stable at 75% or above.

### Example 3

L. rhamnosus in canola was ball milled using Roalox Alumin-fortified Grinding Jars produced by U.S. Stoneware of East Palestine Ohio. The jars were loaded with either 3 mm spherical alumina beads or cylindrical alumina grinding media that was 0.25″ long with an outside diameter of 0.25″. The roller table used had an adjustable speed range of from about 20 to about 300 rpm.

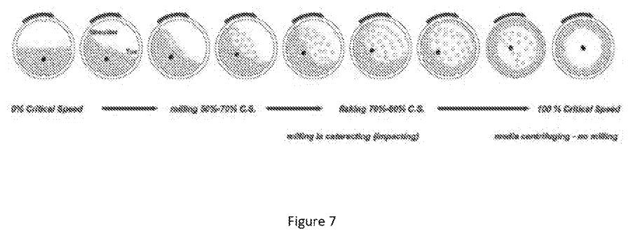

The speed of the ball mill achieves the appropriate particalization. As can be seen in FIG. 7, ball milling speed is often referenced in terms of the percent of critical speed that the process should be run at. FIG. 7 illustrates the effect on the media as the speed of the ball mill is increased from zero to 100% of critical speed. At zero, the media just sits in the jar. At 100% of critical speed, the media is forced against the outside of the jar by centrical force. Most ball milling occurs in the range of 55% to 75% of critical speed. As will be seen in the following results, the ball milling speed can have a big influence on probiotic survival.

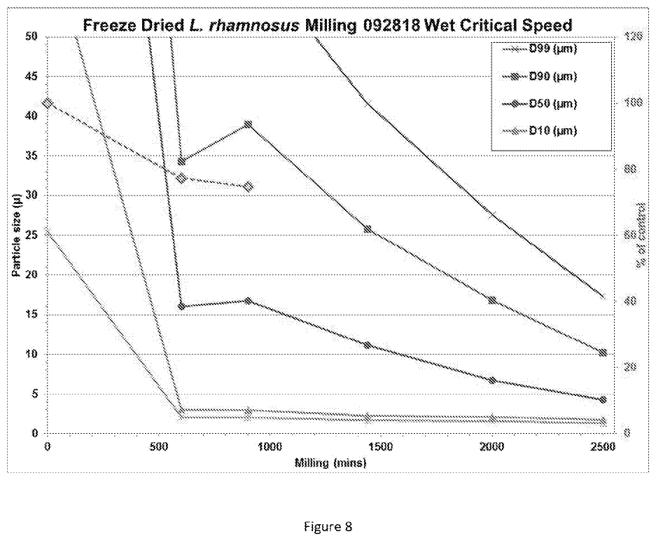

As can be seen in FIG. 8, when L. rhamnosus in canola was subjected to ball milling. Ground probiotics for use in the products as disclosed have an average diameter of from about 30 microns to about 120 microns thereby assuring bacterial survival rate of on the order of 70% or greater.

### Example 4

Microcapsules were produced by ball milling L. rhamnosus in trehalose to a D₅₀ of 7.1. The milled probiotic material was coated with dry canola oil and then provided with a protective outer coating of Tung oil. The microcapsule was cured at 35 degrees C. Samples crushed on artificial skin were compared to uncrushed samples. Uncrushed microcapsules were stored in a water based broth for 48 hours. Osmotic pressure exerts an enormous force on the microcapsules and as a result caused all samples to be either stable in water or not stable. The samples of this example were found to be water stable. Bacterial counts were taken in both the crushed and the uncrushed samples and all were found to continue to include substantial amounts of live bacteria available to colonize on the skin sample.

### Example 5

Formulations and uses for the probiotic microcapsules will be discussed below. Examples of skin care lotions are set forth below in Tables 4 to 7. Examples of hand cleaner, hand soap and shampoo can be found in Tables 8, 9, and 10, respectively. In addition to the formulations described, the probiotics may be incorporated into any art recognized aqueous or non-aqueous based product in which the microcapsule remains stable.

TABLE 4

| INCI Name | Solids (wt. %) |
|---|---|
| Water | 84.50 |
| Glycerin | 5.00 |
| Disodium EDTA | 0.10 |
| Carbomer | 0.40 |
| Acrylates/C10-30 Alkyl Acrylate | 0.80 |
| Crosspolymer | |
| Isopropyl Myristate | 1.50 |
| Caprylate triglyceride | 3.00 |
| Cetyl Alcohol | 2.00 |
| Stearyl alcohol | 1.00 |
| Phenoxyethanol | 0.90 |
| NaOH | 0.80 |

### Additional Ingredients

| Ingredient | Reasons | Solids (wt. %) |
|---|---|---|
| Silicones: Cyclomethicone, dimethicone, or | Emolliency, reduced water activity | 0.5-5% |
| Cyclopentasiloxane | | |
| Humectant: Glycerin, glycols, or triethyl citrate | Emolliency, reduced water activity | 0-10% |
| Prebiotic sugar: Dried glucose syrup, trehalose, | | 0-10% |
| fructose, mannose | | |
| Oligosaccharides: fructo-oligosaccharides, | Reduced water activity, | 0-10% |
| galactooligosaccharises, mannan | reactivation of probiotic | |
| oligosaccharide-rich yeast cell wall | | |
| Powders (PMMA | Powdery afterfeel/drying | 0-5% |

TABLE 5
Example Lotion Formulation (Oil-in-Water #2)

| INCI Name | Solids (wt. %) |
|---|---|
| Water | qs |
| Sodium Polyacrylate | 0.40 |
| Glycerin | 4.00 |
| Caprylic/Capric Triglyceride | 5.00 |
| Cetearyl Alcohol/Ceteareth-20 | 3.00 |
| Polyacrylate-13, Piolyisobutene, | 1.00 |
| Polysorbate 20 | |
| Preservative | optional |

### Additional Ingredients

| Ingredient | Reasons | Solids (wt. %) |
|---|---|---|
| Silicones: Cyclomethicone, dimethicone, or | Emolliency, reduced water activity | 0.5-3% |
| Cyclopentasiloxane | | |
| Humectant: Glycerin, glycols, or triethyl citrate | Emolliency, reduced water activity | 2-5% |
| Prebiotic sugar: Dried glucose syrup, trehalose, | | 1-2% |
| fructose, mannose | | |
| Oligosaccharides: fructo-oligosaccharides, | Reduced water activity, | 0-10% |
| galactooligosaccharises, mannan | reactivation of probiotic | |
| oligosaccharide-rich yeast cell wall | | |
| Powders (PMMA | Powdery afterfeel/drying | 0.5 to- 5% |

TABLE 6
Example Lotion Formulation (Water-in-Oil #1)

| INCI Name | Solids (wt. %) |
|---|---|
| Water | qs |
| Glycerin | 10.0 |
| Sodium Chloride | 1.0 |
| Dimethicone and Dimethiconol | 5.0 |
| Stearoxytrimethylsilane (and) | 2.0 |
| Stearyl Alcohol | |
| Cyclopentasiloxane | 10.0 |
| Mineral Oil | 10.0 |
| Lauryl PEG/PPG-18/18 Methicone | 2.0 |
| Preservative | qs |

TABLE 7
Example Lotion Formulation (Water-in-Oil #2)

| INCI Name | Solids (wt. %) |
|---|---|
| Water | qs |
| Glycerin | 4.00 |
| Magnesium Sulfate | 0.75 |
| (Heptahydrate) | |
| PEG-30 Dipolyhydroxystearate | 1.50 |
| Propylene Glycol Isostearate | 7.50 |
| Isopropyl Isostearate | 7.50 |
| Stearyl alcohol | 2.00 |
| Preservative | qs |

TABLE 8
Waterless Hand Cleaner Example

| Ingredient | Solids (wt %) |
|---|---|
| 1,3 Propane diol | 35.0 |
| Tall Oil | 6.0 |
| Lanolin | 4.0 |
| Tween 20 | 4.0 |
| Propylene Glycol | 0.2 |
| Perfume | 0.1 |
| Water | qs |
| Prebiotic Sugar | 20.0 |
| Chelating Agent | 0.2 |
| NaOH | 0.6 |
| Preservative | qs |

TABLE 9
Hand Soap Example

| Ingredient | Solids (wt %) |
|---|---|
| Water | qs |
| Sodium Laureth Sulfate | 10.0 |
| Cocamidopropyl betaine | 8.0 |
| Sodium Chloride | 2.1 |
| Glycerin | 5.0 |
| Zemea | 5.0 |
| Sodium Lauryl Sulfate | 1.0 |
| Glycol Stearate | 0.5 |
| Sodium Lauroyl lactate | 0.5 |
| fragrance | 0.2 |
| Kathon CG | qs |
| NaOH | qs to pH 5.5 |
| Dye | 0.0000001 |

TABLE 10
Shampoo Example:

| Ingredient | Solids (wt %) |
|---|---|
| Water | qs |
| Cocamidopropyl betaine | 15.0 |
| Sodium laureth sulfate | 4.0 |
| Sodium lauryl sulfate | 4.0 |
| Glycol Distearate | 5.0 |
| Probiotic sugar | 20.0 |
| 1,3 Propane Diol | 10.0 |
| Cocamide MEA | 5.0 |
| Dimethicone | 2.0 |
| Citric Acid | 1.0 |
| Sodium xylene sulfoneate | 0.5 |
| Tetrasodium EDTA | 0.1 |
| Kathon CG | qs |
| NaOH | qs to pH 5.5 |
| Dye | 0.0000001 |

### Example 6—Prophetic Example

The encapsulated probiotic product as described can be delivered to the gut of a user by incorporation of the encapsulated probiotic into or onto a food source. According to this Example, microencapsulated probiotic is sprinkled onto a food, such as bread or pastry and is consumed by the user. The microcapsules should protect the probiotic until it can be released and provide benefit to the user.

Additionally, other embodiments will be apparent from consideration of the specification and practice of the present disclosure. It is intended that the specification and examples be considered as exemplary only, with a true scope and spirit of the invention being indicated by the following claims.

What is claimed is:

1. A microcapsule comprising:
at least one stasis pod comprising at least one freeze-dried probiotic and at least one adjuvant;
a nutrient-rich carrier or polymer core surrounding the at least one stasis pod; and
a protective layer surrounding the nutrient-rich carrier.

2. The microcapsule of claim 1, wherein the probiotic is chosen from one or more of Bifidobacterium, Pediococcus, Leuconostoc, Micrococcus, Escherichia, Staphylococcus, Streptococcus, Candida, and Bacillus.

3. The microcapsule of claim 1, the freeze-dried probiotic further comprises a carrier in which it was freeze-dried.

4. The microcapsule of claim 3, wherein the freeze-dried probiotic and carrier are in the form of particles having a size of from about 2 microns to about 120 microns.

5. The microcapsule of claim 1, wherein the adjuvant comprises one or more waxes or solid support carriers.

6. The microcapsule of claim 4, wherein the stasis pods are surrounded by a nutrient-rich carrier chosen from one or more of shea butter, cocoa butter, coconut oil, or flaxseed oil, vegetable oil, silicone oil, and mineral oil.

7. The microcapsule of claim 1, wherein the protective layer is polymethylene urea or drying oil.

8. The microcapsule of claim 1, wherein a nutrient-rich carrier surrounds the stasis pods and further comprises one or more added nutrients.

9. The microcapsule of claim 8, wherein the added nutrient is cacao.

10. A skin care composition comprising
a microencapsulated probiotic comprising:
at least one stasis pod comprising at least one freeze-dried probiotic and at least one adjuvant;
a nutrient-rich carrier or polymer core surrounding the at least one stasis pod; and
a protective layer surrounding the nutrient-rich carrier; and
a formulation base.

11. The composition of claim 10, wherein the probiotic is chosen from one or more of Bifidobacterium, Pediococcus, Leuconostoc, Micrococcus, Escherichia, Staphylococcus, Streptococcus, Candida, and Bacillus.

12. The composition of claim 10, the freeze-dried probiotic further comprises a carrier in which it was freeze-dried.

13. The composition of claim 12, wherein the freeze-dried probiotic and carrier are in the form of particles having a size of from about 30 microns to about 120 microns.

14. The composition of claim 10, wherein the adjuvant comprises one or more waxes or solid support carriers.

15. The composition of claim 14, wherein the stasis pods are surrounded by a nutrient-rich carrier chosen from one or more of shea butter, cocoa butter, coconut oil, or flaxseed oil, vegetable oil, silicone oil, and mineral oil.

16. The composition of claim 10, wherein the protective layer is polymethylene urea or drying oil.

17. The composition of claim 10, wherein a nutrient-rich carrier surrounds the stasis pods and further comprises one or more added nutrients.

18. The composition of claim 10, wherein the formulation base is for a moisturizing lotion, sunscreen, lip balm, oral care product, shampoo, soap, hair conditioner, baby wipes, perineal wipes, facial cleaning wipes, feminine hygiene pad or tampon, diaper or adult incontinence product, deodorant (roll-on liquid, spray, or stick), pet food, food additives, food condiments, medicinal products, bandages

19. A method for making an encapsulated probiotic comprising:
ball-milling a freeze-dried probiotic in a water saturated oil to a diameter of from about 2 microns to about 120 microns;
mixing the freeze-dried probiotic and an adjuvant to form a stasis pod;
surrounding the stasis pod with a nutrient-rich carrier; and
encapsulating the nutrient-rich carrier and stasis pods in a protective coating.

20. The method of claim 19, wherein the adjuvant is chosen from a wax or a solid support carrier.

21. The method of claim 20, wherein the adjuvant is a wax and the stasis pods are formed by prilling.

22. The method of claim 19, further comprising applying one or more fugitive layers(s).
